(19) United States
(12) Patent Application Publication
(10) Pub. No.: US 2024/0198025 A1
(43) Pub. Date: Jun. 20, 2024

(54) REINFORCED TUBES

(71) Applicant: SMITHS MEDICAL INTERNATIONAL LIMITED, Ashford (GB)

(72) Inventors: JOHN ZAREMBO, Minneapolis, MN (US); Laura Beth Morton, Sevenoaks (GB); Ayesha Bint-E-Siddiq, Folkstone (GB); Christopher John Woosnam, London (GB); Andrew Thomas Jeffrey, Romney Marsh (GB)

(73) Assignee: SMITHS MEDICAL INTERNATIONAL LIMITED, Ashford (GB)

(21) Appl. No.: 18/552,214

(22) PCT Filed: Mar. 22, 2022
(86) PCT No.: PCT/GB2022/000032

§ 371 Date: Sep. 25, 2023

(30) Foreign Application Priority Data
Apr. 19, 2021 (GB) 2105566.0

(51) Int. Cl. A61M 16/04

(52) U.S. Cl. CPC A61M 16/0465; A61M 16/0402; A61M 16/0434; A61M 2205/0216; A61M 2207/10

(57) ABSTRACT

A tracheostomy tube (1) has a shaft (10) with a neck flange (17) dividing the shaft into a patient end (18) and a machine end (19). The shaft is of a silicone material reinforced by a coil (30) embedded within it. The coil (30) comprises multiple closed loops (32) that are longer along the patient end portion than the machine end portion such that the closed ends of the loops contact one another along the machine end portion for maximum strength but leave a longitudinal gap (33) along the patient end portion. An inflation line (14) extends within the wall of the shaft along the gap (33) along the patient end portion (18) and extends externally of the shaft on the machine side of the neck flange (17). The patient end of the inflation line opens into an inflatable sealing cuff (13) extending around the shaft close to the patient end.

This invention relates to reinforced medico-surgical tubes of the kind including a shaft of a flexible plastics material reinforced along a major part of its length by a reinforcement in the form of a coil of a filament of stiff material embedded within the plastics.

Tracheal tubes are used to enable ventilation, respiration, or spontaneous breathing of a patient. Tracheostomy tubes are inserted into the trachea via a surgically formed opening in the neck so that one end locates in the trachea and the other end locates outside the patient adjacent the neck surface. Tracheostomy tubes can be inserted by different techniques, such as the surgical cut-down procedure carried out in an operating theatre or a cricothyroidotomy procedure, which may be carried out in emergency situations.

Tracheostomy tubes are generally used for more long-term ventilation or where it is not possible to insert an airway through the mouth or nose. The patient is often conscious while breathing through a tracheostomy tube, which may be open to atmosphere or connected by tubing to some form of ventilator. The tube is secured in position by means of a neck flange fixed towards the machine end of the tube and positioned to extend outwardly on opposite sides of the tube.

Tracheostomy tubes can be made of various materials and are usually of a bendable plastics material such as PVC, polyurethane, or silicone. Silicone tubes are particularly advantageous for long-term use because they can be highly flexible, making them less traumatic and damaging to tissue contacted by the tube. The soft nature of silicone, however, means that they can be easily kinked and occluded by external pressure or bending unless measures are taken to avoid this. Often, silicone tubes are reinforced by means of a stiff helical member extending along the tube. Typically, the reinforcement member is a metal wire. Tracheostomy tubes are often also provided with an inflatable sealing cuff that is inflated around the tube towards its patient end in order to form a seal with the trachea so that gas is confined to flow along the bore of the tube. The sealing cuff is inflated via an inflation lumen extending along the tube. If the tube is reinforced, the inflation line cannot extend within the thickness of the wall of the tube so extends along the outside of the reinforcement wire. This leads to a protruding ridge extending along the length of the outside of the tube. The problem with this is that the protruding ridge interrupts the smooth outer surface of the tube and can cause discomfort or trauma to tissue in the region of the stoma and the trachea. This can be a particular problem with tubes intended for paediatric and neonatal use.

It has been proposed in GB933307 to incorporate a helical inflation line within the thickness of the wall of the tube to serve the dual purposes of both enabling inflation gas to be supplied to the sealing cuff and acting as a helical reinforcement member. GB2552250 describes a tube having a separate helical reinforcing wire and inflation line wound parallel to the reinforcement. The problem with these arrangements is that, in order to achieve sufficient reinforcement, the inflation line has to be wound with a very close pitch, leading to a large number of turns and a large overall length. The long length of the inflation line gives it a high resistance to gas flow along it, making it difficult to inflate and deflate the sealing cuff. This problem is even worse with tubes, such as those with a silicone cuff, that are inflated using a liquid. In WO2022/018394 there is proposed a reinforcing coil with a C shape in section providing a longitudinal gap along which the inflation line can be extended. Some tubes have a distal portion extending within the body and a proximal portion extending externally of the patient, typically with some form of mounting flange between these two portions by which the tube can be secured with the body. The problem presented by the C-shape coil is that the gap along its length weakens the reinforcement provided by the coil. There are other medico-surgical tubes, as well as tracheal tubes, that need to be flexible and reinforced against kinking or crushing.

It is an object of the present invention to provide an alternative reinforced medico-surgical tube.

According to one aspect of the present invention there is provided a reinforced medico-surgical tube of the above-specified kind, characterised in that the coil has multiple turns formed by loops of the filament, that the loops have opposite closed ends extending around the shaft in opposite directions towards one another and towards a longitudinal locus between the closed ends of the loops, and that the longitudinal locus along a first region of the tube defines a path wider than that along a second region along the tube.

The loops in the coil are preferably longer along the second region than along the first region. The tube may include an elongate member extending within the shaft along the path along the first region but not within the shaft along the second region. The tube preferably includes an inflatable sealing member extending around it towards one end of the first region, the elongate member being an inflation line opening at one end into the sealing member. The filament is preferably of a metal selected from a group including stainless steel, high tensile steel, amalgams, alloys, and Nitinol. Outer arms of loops at opposite ends of the coil may be wider than along the remainder of the coil and have smoothly rounded ends. An outer arm of an end loop of the coil may be linked to an adjacent arm by a bridging piece midway along the length of the outer arm.

According to another aspect of the present invention there is provided a method of making a reinforced medico-surgical tube, characterised in that the method includes the steps of forming a flat coil of a stiff material having multiple loops arranged side by side along the length of the coil, that the coil includes at least a first region and a second region where the width of the coil is different along the two regions, that the method includes the step of bending the coil along its longitudinal axis into a tubular form such as to form a longitudinal gap between the ends of the loops along at least the first region, and subsequently forming a shaft by embedding the coil within a flexible plastics material.

According to a third aspect of the present invention there is provided a tracheostomy tube formed by a method according to the above other aspect of the present invention, characterised in that the tube includes a sealing cuff towards its patient end, a neck flange at a location between a patient end portion of the tube and a machine end portion of the tube, that the first region of the coil extends along the patient end region of the tube leaving a gap along the coil along the patient end region, that the tube includes an inflation tube opening at one end into the sealing cuff, that the inflation tube is embedded within the plastics material and extends along the gap, and that the inflation tube extends externally of the shaft on the machine side of the flange.

The external diameter of the shaft may be greater along the machine end portion of the tube than along the patient end portion of the tube.

According to a fourth aspect of the present invention there is provided a coil for use in reinforcing a medico-surgical tube, characterised in that the coil has multiple turns formed by loops of a filament of a stiff material, that the loops have opposite closed ends, that loops along a first region of the coil are shorter than loops along a second region, such that when the coil is bent into a tubular form along its longitudinal axis to bring the closed ends of the loops of the second region close to one another the closed ends of the loops of the first region are spaced from one another by a gap along the length of the first region.

According to a fifth aspect of the present invention there is provided a tracheostomy tube including a shaft of a flexible plastics material, characterised in that the tube includes a coil according to the above fourth aspect of the invention embedded in the shaft.

A paediatric tracheostomy tube according to the present invention will now be described, by way of example, with reference to the accompanying drawings in which:

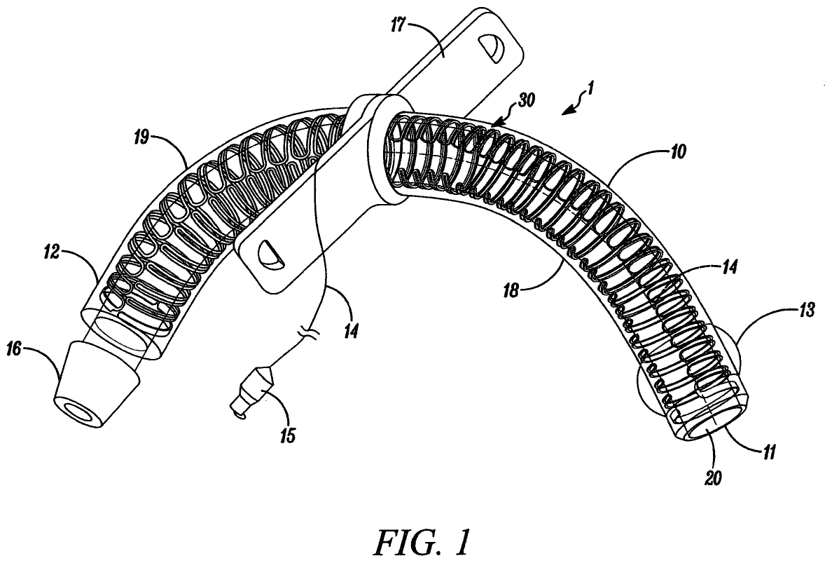

FIG. 1 is a side elevation view of the tracheostomy tube;

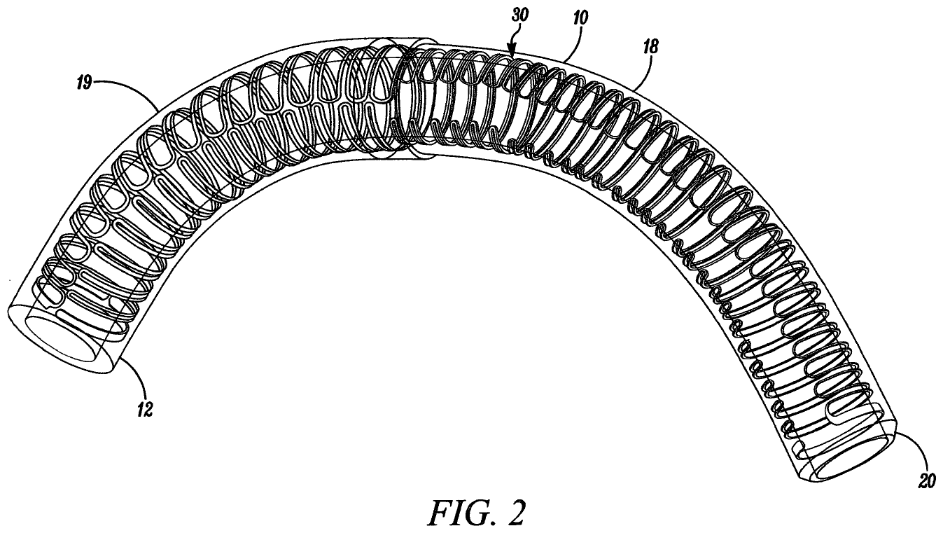

FIG. 2 is perspective phantom drawing of the shaft of the tube;

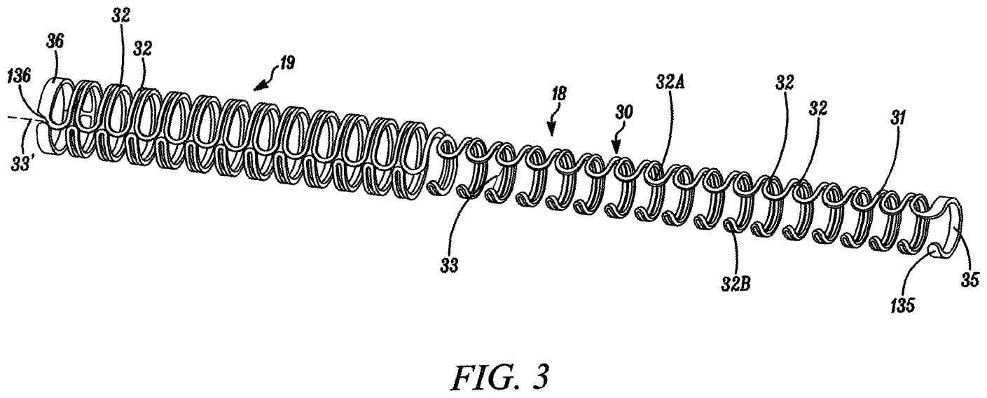

FIG. 3 is a perspective view of the reinforcing coil of the shaft;

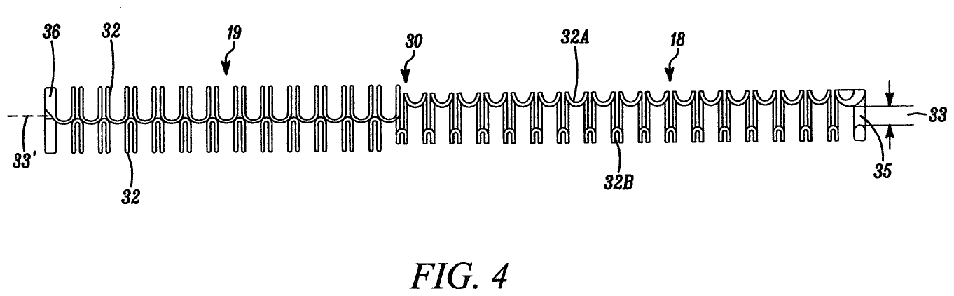

FIG. 4 is a side elevation view of the coil shown in FIG. 3;

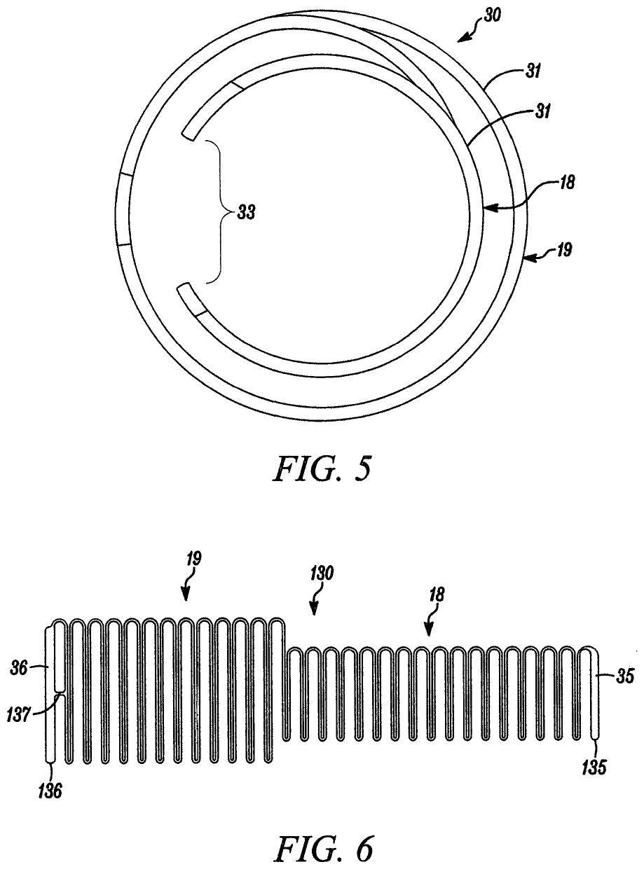

FIG. 5 is an enlarged end view of the coil from its patient end; and

FIG. 6 is a plan view of the coil at a preliminary stage of manufacture

With reference first to FIG. 1, the tracheostomy tube 1 has a curved shaft 10 of circular section with a forward, distal patient end 11 and a rear, proximal machine end 12. The patient end 11 is adapted to be located within the trachea of the patient and the machine end 12 is adapted to extend through the tracheostomy stoma and to be located externally of the patient. Towards its patient end 11 the shaft 10 supports a conventional inflatable sealing cuff 13 the interior of which connects with the patient end of a small-bore inflation line or tube 14 extending along the tube 1. The inflation tube 14 is terminated at its machine end by a conventional combined inflation indicator and valved connector 15 by which inflation fluid such as air or water can be supplied to inflate the cuff 13.

The shaft 10 and cuff 13 are both formed of a soft silicone material, the shaft being reinforced along its length by a reinforcement 30 that will be described in detail later. The machine end 12 of the shaft 10 is terminated by a conventional male tapered connector 16 adapted to make a gas-tight connection in a mating female tapered connector (not shown) at one end of ventilation tubing, or to be left open when the patient is breathing spontaneously. The tube 1 is shown as having a radially extending neck flange 17 located about midway along the length of the shaft 10 and dividing the shaft into a forward, patient end portion 18 and a rear, machine end portion 19 of approximately equal lengths. The patient end portion 18, which extends through the neck tissue of the tracheostomy stoma and into the trachea, has a relatively small external diameter, so that the size of the stoma is minimized. The rear end portion 19 has a slightly larger external diameter than the patient end portion 18. The bore 20 along the shaft 10 is constant along its length to avoid any internal step, giving the rear portion 19 a thicker wall and increased strength. This configuration enables the connection with the machine end 12 of the tube to be isolated from the stoma, to reduce movement at the stoma during connection and disconnection. The increased strength of the rear portion 19 helps better support the weight of tubing connected to the tracheostomy tube.

With reference now also to FIGS. 2 to 5, the reinforcement 30 will be described in greater detail. The reinforcement 30 takes the form of a single-piece coil of a stiff material such as a metal, typically 300 series stainless steel, high tensile steel, amalgams, or alloys such as Nitinol. It would also be possible to use some stiff non-metals such as plastics. Plastics capable of withstanding autoclaving or reprocessing at 121° C. would be particularly useful. The coil 30 is formed by a continuous element or filament 31 wound backwards and forwards in boustrophedon or serpentine fashion into a series of loops 32 extending circumferentially around a part at least of the circumference of the coil. The series of loops comprises alternate wide loops 32A extending in one sense around a part only of the circumference the coil and narrow loops 32B extending in the opposite sense around a part of the circumference of the coil towards the wide loops. The diameter of the coil 30 along the forward portion 18 of the shaft 10 is less than that along the rear portion 19, the loops 32 along the forward portion extending around only an incomplete circumference, thereby leaving a gap 33 between opposite ends of the loops of about 60°. This gap 33 provides a path to accommodate the patient end of the inflation line 14, the machine end of which projects from the neck flange and extends externally of the shaft 10. The loops 32 of the coil 30 along the rear portion 19 are longer than those along the forward portion 18 and the wide and narrow loops are wrapped around a part of the circumference towards one another and into a circular shape having a larger diameter than the loops along the forward portion. The loops 32 along the rear portion 19 also differ from those along the forward portion 18 in that the opposite ends of the adjacent loops contact one another or are closely spaced from one another so that the gap 33′ along this part of the coil 30 is narrower than that along the forward portion 18 or is a closed gap. At opposite ends of the coil 30 the outer arms 35 and 36 of the end loops 32 are wider than along the remainder of the coil so that they can be formed with smoothly rounded ends 135 and 136 to avoid sharp ends that could damage the overlying plastics material. The outer arm 36 of the end loop 32 in the rear portion 19 is also linked to the adjacent arm of the loop by a bridging piece 137 (FIG. 6) midway along its length. This helps ensure that this end arm remains concentric with the adjacent arm when the coil is bend to its circular configuration.

The arrangement described above enables reinforcement with different properties along different parts of a tube to be provided by a single reinforcing element. In particular, it enables a tube to be provided with a gap in the reinforcing element along a part of the tube and not in another part of the tube or with gaps of different widths in different parts of the tube. It also enables reinforcement of a tube with different diameters along its length.

The tube 1 is made using a preliminary form 130 of the reinforcement as shown in FIG. 6 where it is of a flat configuration formed of a rear region 19 of long loops and a forward region 18 of shorter loops all extending in a flat plane. This flat preliminary form can be made in any conventional manner such as by machining from a continuous sheet such by a mechanical, radiation or chemical technique. Alternatively, it could be made by a printing technique such as involving a lithographic or additive process. The next step is to roll up the flat preliminary form 130 into the tubular form shown in FIGS. 3, 4 and 5 by wrapping it about a suitable former defining the desired inner surface of the coil. A reinforcing coil can be formed in this way at relatively low cost and with little wasted material. The tubular coil is then placed in a suitable mould (not shown) defining the internal and external shape of the shaft 10 together with the inflation line 14 extending along the gap along the forward portion 18. The mould is then filled with the liquid silicone or other plastics forming the wall of the shaft and left to cure. After removal from the mould an opening is cut through the outside of the shaft 10 into the forward end of the embedded inflation line 14 and the sealing cuff 13 is attached to the outside of the shaft over this opening. The neck flange 17 and machine end connector 16 are then added to complete the tube.

The arrangement described enables a tube to be provided that is both reinforced against crushing and kinking and that has a cuff inflation lumen enclosed within the wall thickness so that the outer surface can be smooth and uninterrupted. The arrangement, moreover, enables a gap for the inflation lumen to be provided along the inner part of the tube within the patient whilst along the outer part of the tube, where the inflation line can extend externally, the coil can have a smaller or closed gap to provide the maximum reinforcement. Tubes with a diameter as small as 2.5 mm or as large as 9.0 mm could be manufactured in this way.

The reinforcement element need not extend along the entire length of the tube. In some tracheostomy tubes it may be preferable for the tube to be reinforced only along that part of its length that extends through the tracheostomy stoma. A short region at the patient end of the tube could be left unreinforced to provide a soft tip. The rear end of the tube could be left unreinforced to make it easier to insert and retain a tapered coupling. The invention could also have application to reinforced tubes other than tracheal tubes that have an inflatable cuff, such as urethral catheters or vascular dilatation catheters.

1-12. (canceled)

13. A reinforced medico-surgical tube including a shaft of a flexible plastics material reinforced along a major part of its length by a reinforcement in the form of a coil of a filament of stiff material embedded within the plastics, characterised in that the coil has multiple turns formed by loops of the filament, that the loops have opposite closed ends extending around the shaft in opposite directions towards one another and towards a longitudinal locus between the closed ends of the loops, and that the longitudinal locus along a first region of the tube defines a path wider than that along a second region along the tube.

14. A tube according to claim 13, characterised in that the loops in the coil are longer along the second region than along the first region.

15. A tube according to claim 13, characterised in that the tube includes an elongate member extending within the shaft along the path along the first region but not within the shaft along the second region.

16. A tube according to claim 15, characterised in that the tube includes an inflatable sealing member extending around it towards one end of the first region, and that the elongate member is an inflation line opening at one end into the sealing member.

17. A tube according to claim 13, characterised in that the filament is of a metal selected from a group including stainless steel, high tensile steel, amalgams, alloys, and Nitinol.

18. A tube according to claim 13, characterised in that outer arms of loops at opposite ends of the coil are wider than along the remainder of the coil and have smoothly rounded ends.

19. A tube according to claim 13, characterised in that an outer arm of an end loop of the coil is linked to an adjacent arm by a bridging piece midway along the length of the outer arm.

20. A method of making a reinforced medico-surgical tube, characterised in that the method includes the steps of: forming a flat coil of a stiff material having multiple loops arranged side by side along the length of the coil, the coil including at least a first region and a second region where the width of the coil is different along the two regions; bending the coil along its longitudinal axis into a tubular form such as to form a longitudinal gap between the ends of the loops along at least the first region; and subsequently forming a shaft by embedding the coil within a flexible plastics material.

21. A tracheostomy tube formed by the method according to claim 20, characterised in that the tube includes a sealing cuff towards its patient end, a neck flange at a location between a patient end portion of the tube and a machine end portion of the tube, that the first region of the coil extends along the patient end region of the tube leaving a gap along the coil along the patient end region, that the tube includes an inflation tube opening at one end into the sealing cuff, that the inflation tube is embedded within the plastics material and extends along the gap, and that the inflation tube extends externally of the shaft on the machine side of the flange.

22. A tracheostomy tube according to claim 21, characterised in that the external diameter of the shaft is greater along the machine end portion of the tube than along the patient end portion of the tube.

23. A coil for use in reinforcing a medico-surgical tube, characterised in that the coil has multiple turns formed by loops of a filament of a stiff material, that the loops have opposite closed ends, that loops along a first region of the coil are shorter than loops along a second region, such that when the coil is bent into a tubular form along its longitudinal axis to bring the closed ends of the loops of the second region close to one another the closed ends of the loops of the first region are spaced from one another by a gap along the length of the first region.

24. A tracheostomy tube including a shaft of a flexible plastics material, characterised in that the tube includes a col according to claim 23 embedded in the shaft.
